(19) United States
(12) Patent Application Publication
(10) Pub. No.: US 2018/0185437 A1
(43) Pub. Date: Jul. 5, 2018

(54) Granular Pharmaceutical Composition

(71) Applicant: Astellas Pharma, Inc., Tokyo (JP)

(72) Inventors: Mare Nishiura, Tokyo (JP); Kenichi Ashihara, Tokyo (JP); Kanehira Imai, Tokyo (JP); Atsushi Muro, Tokyo (JP); Tsukasa Nishikawahara, Tokyo (JP)

(21) Appl. No.: 15/906,786

(22) Filed: Feb. 27, 2018

Related U.S. Application Data
(63) Continuation of application No. 15/654,870, filed on Jul. 20, 2017, now abandoned.
Application No. 15/654,870 is a continuation of application No. 15/376,932, filed on Dec. 13, 2016, now abandoned.
Application No. 15/376,932 is a continuation of application No. 15/138,489, filed on Apr. 26, 2016, now abandoned.
Application No. 15/138,489 is a continuation of application No. 14/348,110, filed on Mar. 28, 2014, now abandoned.
(60) Provisional application No. 61/541,478, filed on Sep. 30, 2011.

(51) Int. Cl. A61K 38/10; A61K 9/16

(52) U.S. Cl. CPC A61K 38/10; A61K 9/1611; A61K 9/1617; A61K 9/1694; A61K 9/1652; A61K 9/1635

(57) ABSTRACT

The present invention relates to a granular pharmaceutical composition obtained by coating a nucleus with: (1) a layer containing a material having a damp-proofing function, and (2) a drug layer containing linaclotide, a pharmaceutically acceptable salt, or a hydrate thereof, and (3) a layer containing a material having a damp-proofing function. Also, the present invention relates to a method for manufacturing the granular pharmaceutical composition obtained by coating the nucleus with (1) the layer containing the material having a damp-proofing function, (2) the drug layer containing the linaclotide, the pharmaceutically acceptable salt, or the hydrate thereof, and (3) the layer containing the material having a damp-proofing function.

### PRIORITY CLAIM

This application is a continuation of, and claims priority under 35 U.S.C. § 120, to U.S. patent application Ser. No. 15/654,870 filed Jul. 20, 2017, which is a continuation of U.S. patent application Ser. No. 15/376,932 filed Dec. 13, 2016, which is a continuation of U.S. patent application Ser. No. 15/138,489 filed Apr. 26, 2016, which is a continuation of U.S. patent application Ser. No. 14/348,110 filed Mar. 28, 2014, which is the United States National Phase filing of PCT/JP2012/075175 filed Sep. 28, 2012, which claims priority under 35 U.S.C. § 119(e) to U.S. Provisional Application No. 61/541,478 filed Sep. 30, 2011. The disclosure of the prior applications is considered part of (and is incorporated by reference in) the disclosure of this application.

### FIELD OF THE INVENTION

The present invention relates to a granular pharmaceutical composition comprising linaclotide, a pharmaceutically acceptable salt thereof, or a hydrate of either, as well as a method of producing the same.

### BACKGROUND ART

Formulations of many therapeutic polypeptides are prepared in the form of aqueous solutions. This is due to maximum activity being demonstrated by this form. However, almost all polypeptides are not particularly stable in aqueous solution, and such preparations often have short half-lives, necessitating frozen storage. Although aqueous polypeptide solutions can be dried using methods such as freeze drying, spray drying, or other methods, such dried preparations are also unstable, and may be less active than aqueous polypeptide solutions. Typically degradation mechanisms seen in both aqueous solutions and dried preparations include flocculation, oxidation, and hydrolysis. According, almost all therapeutic polypeptides, whether in the form of an aqueous solution or a dried preparation, are stored in a frozen state due to their limited stability.

Linaclotide is a peptide having the amino acid sequence:

(sequence No. 1)
Cys1 Cys2 Glu3 Tyr4 Cys5 Cys6 Asn7 Pro8 Ala9 Cys10
Thr11 Gly12 Cys13 Tyr14.

Linaclotide, which can be orally administered, is useful in treating gastrointestinal disorders and symptoms such as irritable bowel syndrome (IBS) and chronic constipation (CC). Some drug preparations containing linaclotide must be stored frozen in order to avoid breaking down over extended periods of time. However, frozen storage is inconvenient both in terms of the commercial distribution of the drug and storage on the part of patients. Accordingly, there is a demand for a solid linaclotide preparation exhibiting improved stability even at room temperature.

In connection with this peptide, patent document 1 discloses a pharmaceutical composition containing a specific peptide capable of activating guanylate cyclase C (GC-C) receptors in order to provide a composition and method for treating various disorders such as gastrointestinal obstruction, obesity, congestive heart failure, and benign prostatic hyperplasia.

Patent documents 2-5 disclose compositions containing a pharmaceutically acceptable carrier, peptide, and cation and/or amine in order to improve peptide stability against typical degradation mechanisms such as flocculation, oxidation, and hydrolysis.

There remains a demand for a pharmaceutical composition of even more improved stability and efficacy.

### PRIOR ART LITERATURE

### Patent Literature

Patent document 1: Pamphlet of International Publication WO 2004/069165 (specification of U.S. Pat. No. 4,584,911)
Patent document 2: Pamphlet of International Publication WO 2010/027404
Patent document 3: Pamphlet of International Publication WO 2010/019266
Patent document 4: Pamphlet of International Publication WO 2010/027405
Patent document 5: Pamphlet of International Publication WO 2011/017502

### SUMMARY OF THE INVENTION

### Problem to be Solved by the Invention

There is room for improvement in order to provide an even more stable linaclotide-containing pharmaceutical composition.

### Means of Solving the Problem

As the result of dedicated research in order to solve the abovementioned problem, the inventors discovered a stable pharmaceutical composition containing linaclotide, a pharmaceutically acceptable salt thereof, or a hydrate of either, as well as a method of producing the same.

A granular pharmaceutical composition according to the present invention is characterized in having a three-layered structure in which a core is coated with a layer containing a substance exhibiting moisture-proofing action, a drug layer, and a layer containing a substance exhibiting moisture-proofing action, a layer containing a substance exhibiting moisture-proofing action being applied as layers to the inside and outside of a linaclotide drug layer.

The production method according to the present invention is characterized in comprising a drying step in order to adjust the moisture content of the composition according to the present invention during a granulation step.

Specifically, the present invention relates to:

[1] a granular pharmaceutical composition obtained by coating a core with (1) a layer containing a substance exhibiting moisture-proofing action, (2) a drug layer containing linaclotide, a pharmaceutically acceptable salt thereof, or a hydrate of either, and (3) a layer containing a substance exhibiting moisture-proofing action;
[2] the granular pharmaceutical composition according to [1], wherein the layer (1) or (3) containing a substance exhibiting moisture-proofing action contains a substance having a moisture vapor transmission rate of no more than 20 g/(m²·h) as the substance exhibiting moisture-proofing action;
[3] the granular pharmaceutical composition according to [1] or [2], wherein the layer (1) or (3) containing a substance exhibiting moisture-proofing action contains one or more substances selected from the group consisting of polyvinyl alcohol, methacrylate copolymer S, PVA copolymer, aminoalkyl methacrylate copolymer E, methacrylate copolymer LD, and ethyl cellulose as the substance exhibiting moisture-proofing action;
[4] the granular pharmaceutical composition according to any one of [1]-[3], wherein the layer (1) or (3) containing a substance exhibiting moisture-proofing action contains one or more substances selected from the group consisting of polyvinyl alcohol, methacrylate copolymer S, and PVA copolymer as the substance exhibiting moisture-proofing action;
[5] the granular pharmaceutical composition according to any one of [1]-[4], wherein the composition contains at least 100% by weight and no more than 50,000% by weight of the substance exhibiting moisture-proofing action with respect to the weight of the linaclotide, pharmaceutically acceptable salt thereof, or hydrate of either;
[6] the granular pharmaceutical composition according to any one of [1]-[5], wherein the composition contains at least 0.5% by weight and no more than 30% by weight of the substance exhibiting moisture-proofing action with respect to the weight of the core;
[7] a drug preparation containing the granular pharmaceutical composition according to any one of [1]-[6], formed via granulation;
[8] the drug preparation according to [7], wherein the drug preparation contains at least 2.7 μg and no more than 6 mg of linaclotide;
[9] the drug preparation according to [7] or [8], wherein the drug preparation contains one or more substances selected from the group consisting of hydroxypropyl cellulose, hydroxypropyl methyl cellulose, polyvinyl alcohol, PVA copolymer, trehalose, sorbitol, lactitol, isomalt, maltose, oligosaccharides, and maltitol as a binder used for granulation;
[10] the drug preparation according to any one of [7]-[9], wherein the granulated product has a moisture content of at least 0.3% and no more than 4%;
[11] the drug preparation according to any one of [7]-[10], wherein the drug preparation is selected from the group consisting of powders, fine granules, dry syrups, capsules, tablets, orally disintegrating tablets, pills, and troches;
[12] the granular pharmaceutical composition or drug preparation according to any one of [1]-[11], wherein the total amount of degradation products with respect to the linaclotide, pharmaceutically acceptable salt thereof, or hydrate of either, or degradation products thereof, is no more than 8%, the total amount of Cys¹-IMD is no more than 2%, and the total amount of Cys¹-ketone is no more than 2%;
[13] a method of producing a granular pharmaceutical composition, the method comprising (1) a step of coating a core with a layer containing a substance exhibiting moisture-proofing action, (2) a step of coating with a drug layer containing linaclotide, a pharmaceutically acceptable salt thereof, or a hydrate of either, and (3) a step of coating with a layer containing a substance exhibiting moisture-proofing action;
[14] the method of producing a granular pharmaceutical composition according to [13], wherein the layer (1) or (3) containing a substance exhibiting moisture-proofing action contains a substance having a moisture vapor transmission rate of no more than 20 g/(m²·h) as the substance exhibiting moisture-proofing action;
[15] the method of producing a granular pharmaceutical composition according to [13] or [14], wherein the layer (1) or (3) containing a substance exhibiting moisture-proofing action contains one or more substances selected from the group consisting of polyvinyl alcohol, methacrylate copolymer S, PVA copolymer, aminoalkyl methacrylate copolymer E, methacrylate copolymer LD, and ethyl cellulose as the substance exhibiting moisture-proofing action;
[16] the method of producing a granular pharmaceutical composition according to any one of [13]-[15], wherein the layer (1) or (3) containing a substance exhibiting moisture-proofing action contains one or more substances selected from the group consisting of polyvinyl alcohol, methacrylate copolymer S, and PVA copolymer as the substance exhibiting moisture-proofing action;
[17] the method of producing a granular pharmaceutical composition according to any one of [13]-[16], wherein the composition contains at least 100% by weight and no more than 50,000% by weight of the substance exhibiting moisture-proofing action with respect to the weight of the linaclotide, pharmaceutically acceptable salt thereof, or hydrate of either;
[18] the method of producing a granular pharmaceutical composition according to any one of [13]-[17], wherein the composition contains at least 0.5% by weight and no more than 30% by weight of the substance exhibiting moisture-proofing action with respect to the weight of the core;
[19] a method of producing a drug preparation containing the granular pharmaceutical composition according to any one of [13]-[18], the method further containing a granulation step;
[20] the method of producing a drug preparation according to [19], wherein the drug preparation contains at least 2.7 μg and no more than 6 mg of linaclotide;
[21] the method of producing a drug preparation according to [19] or [20], wherein the drug preparation contains one or more substances selected from the group consisting of hydroxypropyl cellulose, hydroxypropyl methyl cellulose, polyvinyl alcohol, PVA copolymer, trehalose, sorbitol, lactitol, isomalt, maltose, oligosaccharides, and maltitol as a binder used for granulation;
[22] the method of producing a drug preparation according to any one of [19]-[21], wherein granules are formed via an intermittent spray method; and
[23] the method of producing a drug preparation according to any one of [19]-[22], wherein the granulated product has a moisture content of at least 0.3% and no more than 4%.

### Effects of the Invention

The present invention is capable of providing a linaclotide-containing pharmaceutical composition of improved stability.

The adoption of a structure in which a core of hygroscopic crystalline cellulose is coated with a layer containing a substance exhibiting moisture-proofing action yields the effect of suppressing reaction with moisture contained in the crystalline cellulose.

The adoption of a structure in which a drug layer is coated with a layer containing a substance exhibiting moisture-proofing action yields the effect of suppression reaction between humidity present in the external environment and the drug layer.

### Preferred Mode for Carrying Out the Invention

An embodiment of the present invention will be described in detail hereafter.

In the context of the present description, “granular pharmaceutical composition” refers to a granular composition containing one or more types of pharmaceutical additive and a drug administered orally in various forms.

In the context of the present description, “core” refers to a substrate capable of forming a pharmaceutically acceptable grain. The core forms a constituent part of the granular pharmaceutical composition according to the present invention, and is a substrate upon which a coating of the coating substance used in the present invention is formed. Crystalline cellulose (in granular form), for example, can be used as the core. The size of the core can be, for example at least 1 μm and no more than 1,000 μm, or, in another aspect, at least 5 μm and no more than 500 μm.

In the context of the present description, “layer containing a substance exhibiting moisture-proofing action” refers to a stability-improving membrane that prevents the drug from reacting with water or humidity and forming a predetermined level or more of degradation products. In addition, said layer is independent and separate from the core and drug layer, and is in a form that serves to prevent moisture contained in the core or external environment from penetrating into the drug layer. Accordingly, the substance exhibiting moisture-proofing action is used, for example, in the drug layer as a binder, and forms in which the substance is mixed with the drug are excluded.

In the context of the present description, “improved stability” refers to the amount of degradation products formed in humid conditions being suppressed and reduced in a form of the preparation including the layer containing a substance exhibiting moisture-proofing action compared to a form of the preparation in which no such layer is present. For example, the total amount of degradation products with respect to the total amount of linaclotide, pharmaceutically acceptable salt thereof, or hydrate of either and degradation products thereof, as measured using high-speed liquid chromatography, is no more than 8%, the amount of Cys¹-IMD is no more than 2%, and the amount of Cys¹-Ketone is no more than 2% after the pharmaceutical composition has been left standing, for example, at 40° C. in conditions of 75% humidity for 6 months, after the pharmaceutical composition has been left standing at 40° C. in conditions of 75% humidity for 3 months in another aspect, after the pharmaceutical composition has been left standing at 40° C. in conditions of 75% humidity for 2 months in yet another aspect, and after the pharmaceutical composition has been left standing at 60° C. for 21 days in still another aspect.

The primary degradation product amounts for Cys¹-IMD and Cys¹-Ketone refer to the degradation product present in the greatest amount (retention time: 1.12 minutes) within the degradation products and the degradation product present in the second greatest amount (retention time: 1.18 minutes) as measured according to the high-speed liquid chromatography method set forth in test example 1.

Linaclotide is a peptide having the amino acid sequence:

(sequence No. 1)
Cys1 Cys2 Glu3 Tyr4 Cys5 Cys6 Asn7 Pro8 Ala9 Cys10
Thr11 Gly12 Cys13 Tyr14.

For more information, see, for example, WHO Drug Information, Vol. 21, No. 3, 2007, page 253, “International Nonproprietary Names for Pharmaceutical Substances”. In the granular pharmaceutical composition according to the present invention, any desired form of linaclotide can be used; examples are a pharmaceutically acceptable salt or hydrate of the peptide, or an isolated and/or purified form of the peptide.

A single salt or a combination of two or more different types of salts can be used.

The granular pharmaceutical composition according to the present invention can be used to treat diseases, disorders, or states that react to treatment using GC-C receptor agonists. The granular pharmaceutical composition according to the present invention can also be used to treat a patient (such as a mammalian animal or a human) suffering from a desired gastrointestinal disorder and/or symptom, or accompanying inflammation or pain. There is no particular limitation upon suitable gastrointestinal disorders or symptoms; examples include irritable bowel syndrome, constipative irritable bowel syndrome, indigestion (including functional dyspepsis and non-ulcerative indigestion), motor dysfunction of the digestive tract, functional gastrointestinal disorders, gastroesophageal reflux disease (GERD), Crohn's disease, ulcerative colitis, inflammatory bowel disease, functional heartburn, gastroparesis, chronic intestinal pseudo-obstruction (or colonic pseudo-obstruction) accompanying constipation, and constipation-related disorders and states, such as chronic constipation, sudden constipation, opioid-induced constipation, and postoperative constipation (postoperative ileus), neuropathic disease-related symptoms, and combinations of these symptoms (such as a combination of irritable bowel syndrome and chronic constipation). In some embodiments, a method of treating gastrointestinal disorders of a patient (such as a mammalian animal or a human) having been diagnosed with one or more gastrointestinal disorders or states, the method involving administration of an effective dose of the composition to the patient, is provided.

The effective dose of a pharmaceutical composition containing linaclotide, a pharmaceutically acceptable salt thereof, or a hydrate of either necessary in order to achieve the desired effects (such as a specific treatment and/or mitigation of symptoms) in the object of treatment depends upon several understood factors, such as the nature and severity of the disorder to be treated, as well as the age, bodyweight, etc. of the patient being treated.

The object or patient for which administration of the granular pharmaceutical composition according to the present invention is an effective treatment of a disease or disorder is preferably a human, but can be any sort of animal, including experimental animals involved in screening or activity experimentation. Accordingly, as will be readily apparent to a person skilled in the art, the method, compound, and composition set forth in the present description is especially suited to administration to a desired animal, especially a desired mammal, including but not limited to a human, a rodent or non-rodent (such as a cat or dog), a domestic animal (including but not limited to a cow, horse, goat, sheep, or pig), a wild animal (whether in the wild or in captivity), a research animal (such as a mouse, rat, rabbit, goat, sheep, pig, dog, or cat), or a bird (such as a chicken, a turkey, or a songbird) (the latter being in cases of veterinary use).

An effective dose of linaclotide for a human adult is, for example, at least 2.7 μg and no more than 6 mg orally administered daily, or, in another aspect, at least 25 μg and no more than 2 mg orally administered daily. In yet another aspect, the dosage level for a human adult is at least 50 μg and no more than 1 mg (for example, 50 μg, 62.5 μg, 100 μg, 125 μg, 150 μg, 200 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, or 1 mg) orally administered daily. In yet another aspect, the dosage level is at least 100 μg and no more than 600 μg orally administered daily, and in yet another aspect, the dosage level is 50 μg, 62.5 μg, 100 μg, 125 μg, 150 μg, 200 μg, 250 μg, 300 μg, 400 μg, 500 μg, or 600 μg orally administered daily. In yet another aspect, the dosage level is 50 μg orally administered daily. In yet another aspect, the dosage level is 62.5 μg orally administered daily. In yet another aspect, the dosage level is 100 μg orally administered daily. In yet another aspect, the dosage level is 100 μg orally administered daily. In yet another aspect, the dosage level is 125 μg orally administered daily. In yet another aspect, the dosage level is 200 μg orally administered daily. In yet another aspect, the dosage level is 250 μg orally administered daily. In yet another aspect, the dosage level is 300 μg orally administered daily. In yet another aspect, the dosage level is 400 μg orally administered daily. In yet another aspect, the dosage level is 500 μg orally administered daily. In yet another aspect, the dosage level is 600 μg orally administered daily.

The daily effective dose of orally administered linaclotide for a child is, for example, at least 0.05 μg and no more than 2 mg; in another aspect, at least 0.05 μg and no more than 100 μg; in yet another aspect, at least 0.1 μg and no more than 90 μg; in yet another aspect, at least 0.1 μg and no more than 50 μg; in yet another aspect, at least 0.1 μg and no more than 25 μg; in yet another aspect, at least 0.1 μg and no more than 10 μg; in yet another aspect, at least 0.1 μg and no more than 5 μg; in yet another aspect, at least 0.1 μg and no more than 1 μg; and in yet another aspect, at least 0.1 μg and no more than 0.5 μg. In yet another aspect, the daily effective dose of orally administered linaclotide for a child is 0.1 μg; in yet another aspect, 0.25 μg; in yet another aspect, 0.5 μg; in yet another aspect, 3.5 μg; in yet another aspect, 15 μg; in yet another aspect, 45 μg; in yet another aspect, 60 μg; and in yet another aspect, 90 μg.

In a certain aspect, the unitary administration form and the daily administered dose are the same. In another aspect, the unitary administration form is administered with food at a desired time of the day, without food at a desired time of the day, or with food after a night of fasting (such as with breakfast). In yet another aspect, the unitary administration form is administered once daily, twice daily, or three times daily. In yet another aspect, 1, 2, or 3 unitary administration forms contain the daily oral dose of linaclotide. Determining the right amount of compound to administer to a patient is the responsibility of the supervising physician. However, the dose used will depend upon many factors, including the age and sex of the patient, the disease being treated, and the severity thereof.

Alternatively, the amount of linaclotide contained in the granular pharmaceutical composition according to the present invention is at least 0.01% by weight and no more than 10% by weight, or, in another aspect, at least 0.05% by weight and no more than 1% by weight.

Alternatively, the amount of linaclotide is at least 2.7 μg and no more than 6 mg per unit of preparation, or, in another aspect, at least 20 μg and no more than 1000 μg.

The crystalline cellulose used in the present invention can be used without limitation as to the bulk density or average polymerization level thereof as long as the cellulose can be used as a core; one example is cellulose obtained by partially depolymerizing and purifying alpha-cellulose obtained as pulp from fibrous plant matter in an acid (Japanese Pharmacopoeia, Sixteenth Edition).

Specific examples of crystalline cellulose include Ceolus PH101, Ceolus PH102, Ceolus PH101D, Ceolus KG802, Ceolus KG801, Ceolus UF711, Ceolus UF702, Ceolus KG1000, Ceolus PH301, Ceolus PH301D, Ceolus PH301Z, Ceolus PH302, Ceolus PHF20JP, Celphere CP102, Celphere CP203, Celphere CP305, and Celphere CP507 (all available from Asahi Kasei); Avicel PH101, Avicel PH112, Avicel PH113, Avicel PH200, Avicel PH301, Avicel PH302, Avicel HFE-102, Avicel DG, and Avicel PH-105 (all available from FMC Biopolymer); Celex 101 (International Specialty Products); Emcocel 90M, Emcocel LM50M, Emcocel 50M, Vivacel 12, VIVAPUR® 101 Premium, VIVAPUR® 105, and VIVAPUR® 301 (J. Rettenmaier & Sohne); Pharmacel 101 (DMV-Fonterra Excipients); and Celphere (San-Ei Gen FFI).

The form of the crystalline cellulose is not limited, and it may be granular, acicular, or the like. Crushed acicular cellulose can also be used. Alternatively, acicular cellulose can be granulated through agitation granulation or high-speed agitation granulation. In another aspect, granular cellulose can be used.

Commercially available mixtures of conjugates of cellulose with other additives (such as carrageenan, sodium carboxymethyl cellulose, guar gum, etc.) can also be used.

Combinations of one or more types of crystalline cellulose of different grades, forms, average particle diameters, or the like can also be used.

There is no particular limitation upon the amount of crystalline cellulose contained in the formulation as long as it is sufficient to create a coating layer containing a substance exhibiting moisture-proofing action.

In a certain aspect, the amount of crystalline cellulose contained in the granular pharmaceutical composition according to the present invention is at least 1% by weight and no more than 99.9% by weight; in another aspect, least 30% by weight and no more than 99% by weight; in still another aspect, at least 80% by weight and no more than 99% by weight; alternatively, the amount is at least 1,000% by weight and no more than 100,000% by weight with respect to the weight of linaclotide; in another aspect, at least 10,000% by weight and no more than 100,000% by weight; and in yet another aspect, at least 10,000% by weight and no more than 50,000% by weight.

There is no particular limitation upon the substance exhibiting moisture-proofing action used in the present invention as long as it is pharmaceutically acceptable, exhibits moisture-proofing action, and suppresses the degradation of the linaclotide, pharmaceutically acceptable salt thereof, or hydrate of either. For example, any substance that suppresses degradation of the linaclotide, pharmaceutically acceptable salt thereof, or hydrate of either by core-derived humidity such as moisture adsorbed to the crystalline cellulose, humidity derived from the production environment, and humidity derived from additives (such as D-mannitol and other saccharides or polymeric substances) or solvents (such as water or methanol) used to perform granulation.

Specific examples include polyvinyl alcohol (“PVA”), methacrylate copolymer S, PVA copolymer, ethyl cellulose and other water-insoluble cellulose ethers, aminoalkyl methacrylate copolymer E, methacrylic acid copolymer L, methacrylate copolymer LD, aminoalkyl methacrylate copolymer RS, methyl acrylate/methyl methacrylate copolymer, and the like. In another aspect, examples include polyvinyl alcohol, methacrylate copolymer S, PVA copolymer, aminoalkyl methacrylate copolymer E, methacrylate copolymer LD, and ethyl cellulose. In yet another aspect, examples include polyvinyl alcohol, methacrylate copolymer S, and PVA copolymer. In yet another aspect, an example is polyvinyl alcohol.

In another aspect, an example is a compound having a moisture vapor transmission rate at a temperature of 23° C.±1° C. and a humidity of 50%±5% RH of no more than 20 g/(m²·h) as measured according to conditions A of JIS Z 0208:1976, “Method for testing the water vapor permeability of moisture-proof packaging materials (inverted cup method)”; in another aspect, a moisture vapor transmission rate of no more than 5 g/(m²·h); and in yet another aspect, a moisture vapor transmission rate of no more than 2 g/(m²·h).

PVA can be used without particular limitation upon the molecular weight, average polymerization level, saponification level, and the like thereof. Specific examples of PVA include Gohsenol™ EG-40, Gohsenol™ EG-40P, Gohsenol™ EG-05, Gohsenol™ EG-05P, and Gohsenol™ NH-17Q (Nippon Synthetic Chemical Industry); and Denka Poval® (Denki Kagaku Kogyo). One or more types of PVA of different grades, viscosities, or the like can be used in combination.

Methacrylate copolymer S is a copolymer of methacrylic acid and methyl methacrylate, and dissolves at pH levels of 7.0 or higher. The chemical name is poly (methacrylic acid-co-methyl methacrylate). A specific example of methacrylate copolymer S is Eudragit® S100 (EVONIK).

The chemical name for PVA copolymer is polyvinyl alcohol/acrylic acid/methyl methacrylate copolymer. PVA copolymer can be used without particular limitation upon the molecular weight, average polymerization level, and the like thereof. Specific examples of PVA copolymer include POVACOAT® R, POVACOAT® L, POVACOAT® F, POVACOAT® FL, and POVACOAT® MP (Daido Chemical).

A specific example of ethyl cellulose is Aquacoat ECD (Dainippon Sumitomo Pharma); specific examples of aminoalkyl methacrylate copolymer E include Eudragit® E100 (EVONIK) and Eudragit® EPO (EVONIK); a specific example of methacrylate copolymer L is Eudragit® L100 (EVONIK); specific examples of methacrylate copolymer LD include Eudragit® L100-55 (EVONIK) and Eudragit® L30D-55 (EVONIK); specific examples of aminoalkyl methacrylate copolymer RS include Eudragit® RL100 (EVONIK), Eudragit® RLP0 (EVONIK), Eudragit® RS100 (EVONIK), and Eudragit® RSP0 (EVONIK); and a specific example of methyl acrylate/methyl methacrylate copolymer is Eudragit® NE30D (EVONIK).

There is no particular limitation upon the amount of substance exhibiting moisture-proofing action in the composition as long as it is ordinarily capable of improving the stability of the linaclotide, pharmaceutically acceptable salt thereof, or hydrate of either. For example, the amount may be at least 0.5% by weight and no more than 30% by weight with respect to the weight of the core, or, in another aspect, at least 1% by weight and no more than 15% by weight. Alternatively, the amount may be at least 100% by weight and no more than 50,000% by weight with respect to the weight of the linaclotide, pharmaceutically acceptable salt thereof, or hydrate of either; in another aspect, at least 500% by weight and no more than 5,000% by weight; and, in yet another aspect, at least 1,000% by weight and no more than 4,000% by weight.

Various pharmaceutical additives can be further added to the granular pharmaceutical composition according to the present invention as desired to create a drug preparation. There is no particular limitation upon these pharmaceutical additives as long as they are pharmaceutically and pharmacologically acceptable. Examples include excipients, binders, stabilizers, antioxidants, disintegrating agents, acidifying agents, foaming agents, artificial sweeteners, flavorings, lubricants, anticaking agents, antimicrobial additives, colorants, buffering agents, and surfactants.

Examples of excipients include crystalline cellulose, D-mannitol, isomalt, sorbitol, dextrose, xylitol, sucrose, starch, lactose, and dextrose.

Examples of binders that can be used in the drug layer include polyvinyl pyrrolidone, polyvinyl alcohol, PVA copolymer, starches (such as cornstarch, potato starch, pre-gelatinized potato starches including Starch 1500® and Starch 1500 LM®, rice starch, wheat starch, and sodium starch glycolate), maltodextrin, gelatin, natural rubber, acacia and other synthetic rubbers, powdered tragacanth, guar gum, cellulose and derivatives thereof (such as methyl cellulose, carboxymethyl cellulose, hydroxyethyl cellulose, hydroxyethyl methyl cellulose, hydroxypropyl cellulose, hydroxypropyl methyl cellulose (hypromellose; products include TC-5E, ETHOCELE 5 Premium LV, etc.) ethyl cellulose, cellulose acetate, calcium carboxymethyl cellulose, sodium carboxymethyl cellulose, crystalline cellulose (including Avicel®-PH-101, Avicel®-PH-103, Avicel®-PH-105, and other Avicel® products from FMC Corporation), methacrylate polymer, cyclodextrin, dextran, polyacrylic acid, chitosan, xanthan gum, polyethylene polypropylene oxide, sodium polyvinyl sulfonate, polyethylene glycol, polyarginine, polycarbophil, polyvinyl pyrrolidone/vinyl acetate copolymer, and poloxamers such as Pluronic®. In another aspect, an example is polyvinyl alcohol.

Examples of binders that can be used for granulation include polyvinyl pyrrolidone, polyvinyl alcohol, PVA copolymer, starches (such as cornstarch, potato starch, pre-gelatinized potato starches including Starch 1500® and Starch 1500 LM®, rice starch, wheat starch, and sodium starch glycolate), maltodextrin, gelatin, natural rubber, acacia and other synthetic rubbers, powdered tragacanth, guar gum, cellulose and derivatives thereof (such as methyl cellulose, carboxymethyl cellulose, hydroxyethyl cellulose, hydroxyethyl methyl cellulose, hydroxypropyl cellulose, hydroxypropyl methyl cellulose (hypromellose) ethyl cellulose, cellulose acetate, calcium carboxymethyl cellulose, sodium carboxymethyl cellulose, crystalline cellulose (including Avicel®-PH-101, Avicel®-PH-103, Avicel®-PH-105, and other Avicel® products from FMC Corporation), methacrylate polymer, cyclodextrin, dextran, polyacrylic acid, chitosan, xanthan gum, polyethylene polypropylene oxide, sodium polyvinyl sulfonate, polyethylene glycol, polyarginine, polycarbophil, polyvinyl pyrrolidone/vinyl acetate copolymer, poloxamers such as Pluronic®, trehalose, sorbitol, lactitol, isomalt, maltose, oligosaccharides, and maltitol. In another aspect, examples include hydroxypropyl cellulose, hydroxypropyl methyl cellulose, polyvinyl alcohol, PVA copolymer, trehalose, sorbitol, lactitol, isomalt, maltose, oligosaccharides, and maltitol. In yet another aspect, examples include polyvinyl alcohol, isomalt, and maltose.

In particular, saccharides such as trehalose, sorbitol, lactitol, isomalt, maltose, oligosaccharides, and maltitol are highly soluble in solvents compared to polymers, allowing for a smaller amount of solvent to be used in preparing the binder solution. In addition, a saccharide-containing binder solution will vaporize faster than a polymer-containing binder solution. In the present invention, a saccharide requiring the use of only small amounts of moisture during the granulation process is preferable, as the drug set forth in the present description will be hydrolyzed by moisture.

Examples of stabilizers include the cations and sterically hindered primary amines set forth in WO/2010/019266.

In another aspect, examples include cations such as magnesium acetate, magnesium chloride, magnesium phosphate, magnesium sulfate, calcium acetate, calcium chloride, calcium phosphate, calcium sulfate, zinc acetate, zinc chloride, zinc phosphate, zinc sulfate, sodium acetate, sodium chloride, sodium phosphate, sodium sulfate, aluminum acetate, aluminum chloride, aluminum phosphate, and aluminum sulfate.

Other examples are amino acids such as histidine, phenylalanine, alanine, glutamic acid, aspartic acid, glutamine, leucine, methionine, asparagine, tyrosine, threonine, isoleucine, tryptophan, and valine.

Examples of antioxidants include butylhydroxy anisole (BHA), butylhydroxy toluene (BHT), vitamin E, benzoic acid, ascorbic acid and pharmaceutically acceptable salts thereof, esters, tocopherol and esters thereof, alpha fatty acids, and beta carotene.

Examples of disintegrating agents include agar, calcium carbonate, crystalline cellulose, croscarmellose sodium, crospovidone, polacrilin potassium, sodium starch glycolate, potato starch, tapioca starch, other starches, gelatinized starch, other types of algin, other types of cellulose, gum, carmellose calcium, carmellose sodium, and low-substituted hydroxypropyl cellulose.

Examples of acidifying agents include citric acid, tartaric acid, and malic acid.

An example of a foaming agent is baking soda.

Examples of artificial sweeteners include sodium saccharin, dipotassium glycyrrhizin, aspartame, stevia, and thaumatin.

Examples of flavorings include lemon, lemon-lime, orange, and menthol.

Examples of lubricants include calcium stearate, magnesium stearate, mineral oil, vegetable oil, glycerol, sorbitol, D-mannitol, polyethylene glycol, other glycols, stearic acid, sodium lauryl sulfate, talc, hydrogenated vegetable oils (such as peanut oil, cottonseed oil, sunflower oil, sesame oil, olive oil, corn oil, and soybean oil), zinc stearate, ethyl oleate, ethyl laurate, agar, silica gel (including SYLOID® and AEROSIL® 200), coagulated aerosol of synthetic silica (Evonik Degussa Co., Plano, Tex., USA), pyrogenic silicone dioxide (CAB-O-SIL, Cabot Co., Boston, Mass., USA), leucine, polyvinyl alcohol, light silicic anhydride, and sucrose fatty esters.

Examples of anticaking agents include calcium silicate, magnesium silicate, silicon dioxide, colloidal silicon dioxide, and talc.

Examples of antimicrobial additives include benzalkonium chloride, benzethonium chloride, benzoic acid, benzyl alcohol, butylparaben, cetylpyridinium chloride, cresol, chlorobutanol, dehydroacetic acid, ethylparaben, methylparaben, phenol, phenylethyl alcohol, phenoxyethanol, phenylmercuric acetate, phenylmercuric nitrate, potassium sorbate, propyl paraben, sodium benzoate, sodium dehydroacetate, sodium propionate, sorbic acid, thimerosal, and thymol.

Examples of colorants include yellow iron oxide, red iron oxide, black iron oxide, Food Yellow 4 and 5, Food Red 3 and 102, and Food Blue 3.

Examples of buffers include citric acid, succinic acid, fumaric acid, tartaric acid, ascorbic acid and salts thereof, glutamic acid, glutamine, glycine, aspartic acid, alanine, arginine and salts thereof, magnesium oxide, zinc oxide, magnesium hydroxide, phosphoric acid, and boric acid and salts thereof.

Examples of surfactants include polysorbate 80, sodium lauryl sulfate, and polyoxyethylene hydrogenated castor oil.

Suitable amounts of one or more types of pharmaceutical additive may be added as necessary.

Any pharmaceutical additives should be used in amounts falling within ranges allowing the desired effects of the present invention to be obtained.

Regarding the ratio of stabilizers (cations and/or primary amines) to linaclotide, the molar ratio of cations to sterically hindered primary amines to linaclotide (for example, of calcium ions to leucine to linaclotide) is, for example, 5-100:5-50:1. The molar ratio of cations to sterically hindered primary amines (for example, of calcium ions to leucine) is either 1:1 or, preferably, greater than 2:1 (for example, 5:1-2:1). According, in a certain aspect, the molar ratio of cations to sterically hindered primary amines to linaclotide (for example, calcium ions to leucine to linaclotide) is 100:50:1, 100:30:1, 80:40:1, 80:30:1, 80:20:1, 60:30:1, 60:20:1, 50:30:1, 50:20:1, 40:20:1, 20:20:1, 10:10:1, 10:5:1, or 5:10:1. If a binder (such as methyl cellulose) is used, an amount of at least 0.5% by weight and no more than 2.5% by weight (such as at least 0.7% by weight and no more than 1.7% by weight, at least 0.7% by weight and no more than 1% by weight, 1.5% by weight, or 0.7% by weight).

The granular pharmaceutical composition according to the present invention can be used in various preparations of the pharmaceutical composition. Examples of preparations of the pharmaceutical composition include powders, fine granules, dry syrups, capsules, tablets, orally disintegrating tablets, pills, and troches. In another aspect, an example is tablets.

A method of producing the pharmaceutical composition according to the present invention will be described hereafter.

The method of producing the granular pharmaceutical composition according to the present invention comprises at least a step of forming a coating of a layer containing a substance exhibiting moisture-proofing action before and after coating the core with a linaclotide-containing drug layer. A barrier layer may be disposed, as desired, between, for example, the drug layer and the layer containing a substance exhibiting moisture-proofing action. The method may also comprise a mixing step, granulation step, molding step, film coating step, or packaging step capable of being performed after the coating step.

### Coating Step

In order to obtain the granular pharmaceutical composition according to the present invention, a coating of the layer containing a substance exhibiting moisture-proofing action is formed before and after coating the core with the drug layer. There is no particular limitation upon the apparatus or means used as long as the method allows for the core to be ordinarily pharmaceutically coated with the layer containing a substance exhibiting moisture-proofing action and the drug layer.

Examples of coating apparatus include fluidized bed coating apparatus, rolling coating apparatus, and centrifugal rolling coating apparatus. In another aspect, an example is a fluidized bed coating apparatus.

For example, while fluidizing the core particles with heated air using a fluidized bed lateral spray coating apparatus, a fluid containing the substance exhibiting moisture-proofing action is injected, as necessary, using a spray gun. Next, a liquid containing the drug and pharmaceutical additives is injected as necessary. Next, a fluid containing the substance exhibiting moisture-proofing action is injected, as necessary. The injection liquid is prepared by dissolving or dispersing the ingredients in a vehicle such as water, ethanol, or methanol. A mixture of these vehicles may also be used, as necessary.

Drying can be performed during and/or after the coating process. There is no particular limitation upon the drying method as long as it is an ordinary method of pharmaceutical drying.

The product temperature when coating the core with the layer containing a substance exhibiting moisture-proofing action or drug layer is at least 20° C. and no more than 70° C., or, in another aspect, at least 30° C. and no more than 60° C.

The granular pharmaceutical composition preferably has a moisture content of no more than 6% during the coating process. In another aspect, the composition has a moisture content of at least 0.3% and no more than 6%, and, in yet another aspect, at least 0.3% and no more than 4%.

Air with adjusted humidity can also be fluidized during coating. For example, air or the like containing moisture at a concentration such the dew point is reached at 14° C. can be used. In another aspect, air having a dew point temperature of at least 12° C. and no more than 16° C. can be used, and, in yet another example, air having a dew point temperature of at least 13° C. and no more than 15° C. can be used.

### Granulation Step

In the present invention, a granulation step is preferably included for the sake of uniformity of content.

The coated product obtained during the coating step and the various additives are introduced into a granulator, and spray with a binder solution.

An example of a granulator is a fluidized bed granulator. Examples of granulation methods include fluidized bed granulation, melt granulation, high-speed agitation granulation, shredding (crushing) granulation, extrusion granulation, rolling granulation, spray granulation, and dry granulation. In another aspect, an example is fluidized bed granulation.

Examples of binders include polyvinyl pyrrolidone, polyvinyl alcohol, PVA copolymer, starches (such as cornstarch, potato starch, pre-gelatinized potato starches including Starch 1500® and Starch 1500 LM®, rice starch, wheat starch, and sodium starch glycolate), maltodextrin, gelatin, natural rubber, acacia and other synthetic rubbers, powdered tragacanth, guar gum, cellulose and derivatives thereof (such as methyl cellulose, carboxymethyl cellulose, hydroxyethyl cellulose, hydroxyethyl methyl cellulose, hydroxypropyl cellulose, hydroxypropyl methyl cellulose (hypromellose) ethyl cellulose, cellulose acetate, calcium carboxymethyl cellulose, sodium carboxymethyl cellulose, crystalline cellulose (including Avicel®-PH-101, Avicel®-PH-103, Avicel®-PH-105, and other Avicel® products from FMC Corporation), methacrylate polymer, cyclodextrin, dextran, polyacrylic acid, chitosan, xanthan gum, polyethylene polypropylene oxide, sodium polyvinyl sulfonate, polyethylene glycol, polyarginine, polycarbophil, polyvinyl pyrrolidone/vinyl acetate copolymer, poloxamers such as Pluronic®, trehalose, sorbitol, lactitol, isomalt, maltose, oligosaccharides, and maltitol. In another aspect, examples include hydroxypropyl cellulose, hydroxypropyl methyl cellulose, polyvinyl alcohol, PVA copolymer, trehalose, sorbitol, lactitol, isomalt, maltose, oligosaccharides, and maltitol. In yet another aspect, examples include polyvinyl alcohol, isomalt, and maltose.

Saccharides such as trehalose, sorbitol, lactitol, isomalt, maltose, oligosaccharides, and maltitol are highly soluble in solvents compared to polymers, allowing for a smaller amount of solvent to be used in preparing the binder solution. In addition, the vehicle of a saccharide-containing binder solution will vaporize faster than that of a polymer-containing binder solution. In the present invention, a saccharide requiring the use of only small amounts of moisture during the granulation process is preferable, as the linaclotide set forth in the present description will be hydrolyzed by moisture.

The binder liquid is prepared by dissolving or dispersing the ingredients in a vehicle such as water, ethanol, or methanol. A mixture of these vehicles may also be used, as necessary.

The conditions under which the spray liquid is prepared may be selected, as appropriate, without any particular limitation, but the liquid preferably has a solids concentration of at least 0.5% by weight and no more than 50% by weight. The higher the solids concentration is, the less moisture is required, contributing to the stability of the drug. A solids concentration exceeding 50% by weight will impede uniform granulation.

The method by which the binder solution is sprayed should promote granule bonding and allow a granulated product to be obtained; examples include continuous spraying, or an intermittent spray method in which a drying step or additional shaking step is provided during the granulation process. In another aspect, an example is an intermittent spray method.

“Intermittent spray method” refers to non-continuous spraying. The intermittent spray method can contain two or three steps selected from spraying, drying, and shaking. One example is a granulating spray method in which a cycle of performing spraying for a predetermined length of time followed by performing drying for a predetermined length of time is repeated. The cycle can be set, as appropriate, when engaging in production. For example, after performing spraying for at least 5 second and no more then 30 seconds, the granulated product is dried at a temperature of at least 30° C. and no more than 40° C. for at least 10 seconds and no more than 50 seconds. If there is no drying time, the vehicle contained in the binder solution will not dry, potentially leading to the degradation of the linaclotide. If the drying time is too long, it will be impossible to form granules, or a granulated product containing fine powder incapable of forming granules will be yielded.

There is no particular limitation upon the amount of sprayed binder solution as long as granules can be formed. For example, an amount equivalent to at least 0.5% by weight and no more than 10% by weight of the pharmaceutical composition containing the granular pharmaceutical composition in terms of solids can be used.

There is no particular limitation upon the spraying speed as long as granules can be formed. For example, the spray speed used to produce 1 kg of granulated product can be at least 6 g/min and no more than 30 g/min. Increasing the amount of sprayed liquid makes it possible to reduce the amount of ungranulated particles (fine powder). In addition, providing a drying step during intermittent granulation allows the moisture content of the granules to be minimized.

Drying can be performed after granulation. Any drying method ordinarily used to perform pharmaceutical drying may be used without particular limitation; examples include circulation drying and vacuum drying. The drying temperature is, for example, at least 40° C. and no more than 90° C., or, in another aspect, at least 50° C. and no more than 80° C. Drying time is, for example, at least 1 minute and no more than 60 minutes, or, in another aspect, at least 5 minutes and no more than 30 minutes.

The product temperature during granulation is preferably at least 30° C. and no more than 60° C., or, in another aspect, at least 40° C. and no more than 50° C. If the product temperature falls below 30° C., the moisture content of the pharmaceutical composition containing the granular pharmaceutical composition during granulation will increase, potentially hastening the degradation of the pharmaceutical composition.

There is no particular limitation upon the moisture content of the (granulated) pharmaceutical composition containing the granular pharmaceutical composition during granulation as long as the stability of the drug is no affected; if, for example, D-mannitol is used as an excipient, the moisture content is preferably no more than 6%; in another aspect, at least 0.3% and no more than 6%; and, in yet another example, at least 0.3% and no more than 4% (as measured using a Mettler-Toledo halogen moisture analyzer at 105° C. for 30 minutes).

Air with adjusted humidity can also be fluidized during granulation. For example, air or the like containing moisture at a concentration such the dew point is reached at 14° C. can be used. In another aspect, air having a dew point temperature of at least 12° C. and no more than 16° C. can be used, and, in yet another example, air having a dew point temperature of at least 13° C. and no more than 15° C. can be used.

### Mixing Step

In the mixing step, the granulated product and the various additives are mixed.

### Molding Step

In the molding step, capsules are filled with the granulated or mixed product to create capsules.

Alternatively, the granulated or mixed product can be compressed and molded using a rotary tablet press to create tablets.

Any apparatus for means may be used without particular limitation in the compression step as long as the pharmaceutical composition according to the present invention is molded. Examples include a direct tablet pressing method in which the coated product and suitable pharmaceutical additives are mixed, followed by performing compression molding to obtain tablets, or a method in which the granulated product is further mixed with a lubricant, after which compression molding is performed to produce tablets.

Examples of tablet pressing devices include rotary tablet presses, single-punch tablet presses, oil presses, and the like. There is no particular limitation upon the pressure and other tablet pressing conditions as long as tablets can be molded and the pressure will not break the tablets during the production process.

### Film Coating Step

A film coating may be applied to the surface of the tablets following pressing, as appropriate.

Any method ordinarily used to pharmaceutically apply film coatings may be used without particular limitation. Examples include pan coating and dip coating.

There is no particular limitation upon the film coating agent as long as it is a substance capable of forming a film coating upon tablets. Examples include sodium carboxymethyl cellulose, cellulose acetate phthalate, ethyl cellulose, gelatin, pharmaceutical glaze, hydroxypropyl cellulose, hydroxypropyl methyl cellulose, hydroxypropyl methyl cellulose phthalate, methyl cellulose, polyethylene glycol, polyvinyl acetate phthalate, shellac, sucrose, titanium oxide, carnauba wax, and microcrystalline wax. Suitable amounts of one or more types of film coating agent may be added as necessary.

There is no particular limitation upon the film coating rate as long as a film is formed on the tablet. For example, a rate of at least 1% by weight and no more than 5% by weight with respect to the weight of the tablet is possible.

Drying may optionally be performed after the film coating is applied. Any method ordinarily used to perform pharmaceutical drying may be used without particular limitation. Suitable drying conditions may be set without particular limitation out of consideration, for example, for the stability of the drug preparation.

### Packaging Step

There is no particular limitation upon the packaging step as long as a package capable of storing the pharmaceutical composition is formed.

Examples of packages include aluminum pouches, aluminum-aluminum blisters, glass bottles, plastic bottles, and the like.

A desiccant may be introduced into the package. Any desiccant can be used without particular limitation as long as it absorbs the moisture within the package.

Examples include silica gel, synthetic zeolite, silica-alumina gel, calcium chloride, calcium carbonate, magnesium carbonate, calcium oxide, calcium hydroxide, montmorillonite, and allophane. In another aspect, examples include silica gel and synthetic zeolite.

Any housing method capable of housing the desiccant may be used without particular limitation.

Examples include layering the dessicant within the package, as in the case of MoistCatch™, housing the desiccant in a breathable pouch, which is disposed within the package, and disposing a desiccant in sheet form within the package.

### WORKING EXAMPLES

The present invention will now be described in further detail with using working examples, comparative examples, and test examples, but the present invention should not be construed as being limited to these examples.

Linaclotide produced according to the method set forth in WO/2004/069165 was used.

### Working Example 1

TABLE 1

| | | |
|---|---|---|
| Core | Crystalline cellulose (granular) | 22.66 mg |
| First layer | Partially saponified polyvinyl alcohol | 0.680 mg |
| Second | Linaclotide | 0.0625 mg |
| layer | Calcium chloride | 0.361 mg |
| | L-Leucine | 0.161 mg |
| | Hypromellose | 0.164 mg |
| Third layer | Partially saponified polyvinyl alcohol | 0.723 mg |

### (1) Preparing First Layer

1,000 g of partially saponified polyvinyl alcohol (trade name: Gohsenol™ EG-05P, obtainable from Nippon Synthetic Chemical Industry; likewise hereafter) was dissolved in 4,000 g purified water. 3,000 g of the partially saponified polyvinyl alcohol solution was sprayed onto 20 kg (granulated) crystalline cellulose (trade name: CP-305; Asahi Kasei Chemicals) using a Glatt GPCG-15 fluidized bed granulation apparatus (likewise for second layer) to prepare granules coated with a first layer.

### (2) Preparing Second Layer

48.06 g linaclotide was dispersed in 7,601 g purified water, after which a suitable amount of 10% hydrochloric acid (Wako Pure Chemicals) was added dropwise thereto to adjust the pH to 1.5-2.0. Separately, 123.8 g L-Ieucine (trade name: L-Leucine, USP; J. T. Baker; likewise hereafter), 277.6 g calcium chloride (trade name: Calcium Chloride (Dihydrate); Spectrum; likewise hereafter) and 126.1 g hypromellose (HPMC) (trade name: ETHOCEL E5 Premium LV; Dow Chemical) were dissolved in 5,858 g of purified water, and 10% hydrochloric acid (Wako Pure Chemicals) was added dropwise thereto to adjust the pH to 1.5-2.0. After the ingredients had completely dissolved, the two liquids were mixed. The mixed fluid was sprayed onto 17.981 kg of granules coated with the first layer using a fluidized bed granulation apparatus to prepare granules coated with a second layer.

### (3) Preparing Third Layer

100 g partially saponified polyvinyl alcohol was dissolved in 900 g purified water. 300 g of the partially saponified polyvinyl alcohol solution was sprayed onto 1,000 g of the granules coated with the second layer using a Glatt GPCG-1 fluidized bed granulation apparatus to coat the granules with a third layer, thereby preparing the granular pharmaceutical composition according to the present invention.

### (4) Filling Capsules

25.3 mg of the granules coated with the third layer were used to fill #2 gelatin capsules to prepare capsules of the granular pharmaceutical composition according to the present invention.

### Working Example 2

TABLE 2

| | | |
|---|---|---|
| Core | Crystalline cellulose (granular) | 22.66 mg |
| First layer | Partially saponified polyvinyl alcohol | 0.680 mg |
| Second | Linaclotide | 0.0625 mg |
| layer | Calcium chloride | 0.361 mg |
| | L-Leucine | 0.161 mg |
| | Partially saponified polyvinyl alcohol | 0.164 mg |
| Third layer | Partially saponified polyvinyl alcohol | 0.723 mg |

TABLE 3

| | | |
|---|---|---|
| Granulation | Coated particles with third layer | 24.81 mg |
| | D-Mannitol | 223.4 mg |
| | Maltose | 12.4 mg |
| Mixing | Sodium starch glycolate | 13.0 mg |
| | Magnesium stearate | 1.3 mg |

### (1) Preparing First Layer

100 g partially saponified polyvinyl alcohol was dissolved in 900 g purified water. 360 g of the partially saponified polyvinyl alcohol solution was sprayed onto 1,200 g (granulated) crystalline cellulose (trade name: CP-102Y; Asahi Kasei Chemicals) using a Glatt GPCG-1 fluidized bed granulation apparatus to prepare granules coated with a first layer.

### (2) Preparing Second Layer

2.937 g linaclotide was dispersed in 363.9 g purified water, after which a suitable amount of 10% hydrochloric acid (Wako Pure Chemicals) was added dropwise thereto to adjust the pH to 1.5-2.0. Separately, 100 g partially saponified polyvinyl alcohol was dissolved in 900 g purified water. Separately, 7.56 g L-leucine and 16.96 g calcium chloride were dissolved in 198.9 g purified water, 77.0 g of the previously prepared partially saponified polyvinyl alcohol solution was added thereto, and 10% hydrochloric acid was added dropwise thereto to adjust the pH to 1.5-2.0. After the ingredients had completely dissolved, the two liquids were mixed. The mixed fluid was sprayed onto 1096.8 g of granules coated with the first layer using a fluidized bed granulation apparatus to prepare granules coated with a second layer.

### (3) Preparing Third Layer

100 g partially saponified polyvinyl alcohol was dissolved in 900 g purified water. 270 g of the partially saponified polyvinyl alcohol solution was sprayed onto 900 g of the granules coated with the second layer using a fluidized bed granulation apparatus to coat the granules with a third layer, thereby preparing granules coated with the third layer.

### (4) Granulation

200 g maltose (trade name: Sunmalt-S; Sanwa Starch; likewise hereafter) was dissolved in 800 g purified water. Using a Glatt GPCG-1 fluidized bed granulation apparatus, 86.9 g of the granules coated with the third layer and 781.9 g D-mannitol (trade name: Pearlitol® 50C, Roquette; likewise hereafter) were mixed in a fluidized bed, and the mixed product was sprayed with 217.0 g of the maltose solution at 10 g/min to create granules (product temperature: roughly 40° C.; spray pressure: 0.08 MPa; air flow rate: 0.2 m³/min). Granulation was performed using an intermittent spray method involving spraying for 15 seconds, drying for 40 seconds, and shaking for 5 seconds. The granulated product was dried until a product temperature of 47° C. was reached to obtain a granulated pharmaceutical composition containing the granular pharmaceutical composition according to the present invention.

### (5) Mixing, Tablet-Making

521.2 g of the granulated pharmaceutical composition containing the granular pharmaceutical composition was mixed with 26 g sodium starch glycolate (trade name: Primojel®, DMV; likewise hereafter) and 2.6 g magnesium stearate (trade name: Parteck® LUB MST, Merck; likewise hereafter) using a polyethylene bag, after which the mixture was compression molded using a Hata Iron Works X-20 rotary tablet press to obtain tablets of the pharmaceutical composition containing the granular pharmaceutical composition according to the present invention.

### Working Examples 3-6

TABLE 4

| | | |
|---|---|---|
| Core | Crystalline cellulose (granular) | 22.66 mg |
| First layer | Partially saponified polyvinyl alcohol | 0.680 mg |
| Second | Linaclotide | 0.0625 mg |
| layer | Calcium chloride | 0.361 mg |
| | L-Leucine | 0.161 mg |
| | Hypromellose | 0.164 mg |
| Third layer | Partially saponified polyvinyl alcohol | 1.72 mg |

TABLE 5

| | | |
|---|---|---|
| Mixing | Coated particles with third layer | 25.8 mg |
| | Various sugars or sugar alcohols | 232 mg |

TABLE 6

| | |
|---|---|
| Working | D-Mannitol |
| Example 3 | |
| Working | Lactitol |
| Example 4 | |
| Working | Maltitol |
| Example 5 | |
| Working | Trehalose |
| Example 6 | |

### (1) Preparing First Layer

1,500 g partially saponified polyvinyl alcohol was dissolved in 13.5 kg purified water. 6000 g of the partially saponified polyvinyl alcohol solution was sprayed onto 20 kg (granulated) crystalline cellulose (trade name: CP-305; Asahi Kasei Chemicals) using a Glatt GPCG-15 fluidized bed granulation apparatus (likewise for second layer) to prepare granules coated with a first layer.

### (2) Preparing Second Layer

48.06 g linaclotide was dispersed in 6,778 g purified water, after which a suitable amount of 10% hydrochloric acid (Wako Pure Chemicals) was added dropwise thereto to adjust the pH to 1.5-2.0. Separately, 123.8 g L-leucine, 277.6 g calcium chloride, and 126.1 g hypromellose (HPMC) (trade name: ETHOCELE 5 Premium LV, Dow Chemical) were dissolved in 5,816 g purified water, and 10% hydrochloric acid (Wako Pure Chemicals) was added dropwise thereto to adjust the pH to 1.5-2.0. After the ingredients had completely dissolved, the two liquids were mixed. The mixed fluid was sprayed onto 17.948 kg of granules coated with the first layer using a fluidized bed granulation apparatus to prepare granules coated with a second layer.

### (3) Preparing Third Layer

100 g partially saponified polyvinyl alcohol was dissolved in 900 g purified water. 714 g of the partially saponified polyvinyl alcohol solution was sprayed onto 1,000 g of the granules coated with the second layer using a Glatt GPCG-1 fluidized bed granulation apparatus to coat the granules with a third layer, thereby preparing the granular pharmaceutical composition according to the present invention.

### (4) Mixing

25.8 mg of the granules coated with the third layer and 232 mg D-mannitol were introduced into a glass bottle, and the bottle was induction sealed to prepare a pharmaceutical composition containing the granular pharmaceutical composition according to the present invention (working example 3).

Lactitol (trade name: Milchen® Fine-powdered, Mitsubishi Shoji Foodtech; likewise hereafter) was substituted for the D-mannitol to prepare a pharmaceutical composition containing the granular pharmaceutical composition according to working example 4. Maltitol (trade name: SweetPearl® P200, Roquette; likewise hereafter) was substituted for the D-mannitol to prepare a pharmaceutical composition containing the granular pharmaceutical composition according to working example 5. Trehalose (trade name: TREHA, Hayashibara; likewise hereafter) was substituted for the D-mannitol to prepare a pharmaceutical composition containing the granular pharmaceutical composition according to working example 6.

### Working Example 7

| | | |
|---|---|---|
| Core | Crystalline cellulose (granular) | 22.66 mg |
| First layer | Partially saponified polyvinyl alcohol | 0.680 mg |
| Second | Linaclotide | 0.0625 mg |
| layer | Calcium chloride | 0.361 mg |
| | L-Leucine | 0.161 mg |
| | Hypromellose | 0.164 mg |
| Third layer | Partially saponified polyvinyl alcohol | 0.723 mg |

TABLE 8

| | | |
|---|---|---|
| Granulation | Coated particles with third layer | 24.81 mg |
| | D-Mannitol | 227.7 mg |
| | Hypromellose | 7.6 mg |

### (1) Preparing First Layer

1,000 g partially saponified polyvinyl alcohol was dissolved in 4,000 g purified water. 3,000 g of the partially saponified polyvinyl alcohol solution was sprayed onto 20 kg (granulated) crystalline cellulose (trade name: CP-305; Asahi Kasei Chemicals) using a Glatt GPCG-15 fluidized bed granulation apparatus (likewise for second layer) to prepare granules coated with a first layer.

### (2) Preparing Second Layer

48.06 g linaclotide was dispersed in 7,601 g purified water, after which a suitable amount of 10% hydrochloric acid (Wako Pure Chemicals) was added dropwise thereto to adjust the pH to 1.5-2.0. Separately, 123.8 g L-leucine, 277.6 g calcium chloride, and 126.1 g hypromellose (HPMC) (trade name: ETHOCEL E5 Premium LV, Dow Chemical) were dissolved in 5,858 g purified water, and 10% hydrochloric acid (Wako Pure Chemicals) was added dropwise thereto to adjust the pH to 1.5-2.0. After the ingredients had completely dissolved, the two liquids were mixed. The mixed fluid was sprayed onto 17.981 kg of granules coated with the first layer using a fluidized bed granulation apparatus to prepare granules coated with a second layer.

### (3) Preparing Third Layer

100 g partially saponified polyvinyl alcohol was dissolved in 900 g purified water. 300 g of the partially saponified polyvinyl alcohol solution was sprayed onto 1,000 g of the granules coated with the second layer using a Glatt GPCG-1 fluidized bed granulation apparatus to coat the granules with a third layer, thereby preparing the granular pharmaceutical composition according to the present invention.

### (4) Granulation

50 g hypromellose (trade name: TC-5E, Shin'etsu Chemical) was dissolved in 450 g purified water. 86.8 g of granules coated with the third layer and 781.5 g D-mannitol were mixed using a Glatt GPCG-1 fluidized bed granulation apparatus, and the mixed product was sprayed with 259 g of the hypromellose solution at a rate of 6 g/min to create granules. Granulation was performed using an intermittent spray method involving spraying for 30 seconds, drying for 20 seconds, and shaking for 10 seconds. The granulated product was dried until 47° C. was reached to obtain a granulated pharmaceutical composition containing the granular pharmaceutical composition according to the present invention. The moisture value of the granulation was at least 0.3% and no more than 0.8%.

Production conditions were as follows.

TABLE 9

| Manufacturing conditions | Working Example 7 | |
|---|---|---|
| Product temperature | 40-44° | C. |
| Air pressure of spray | 0.08 | MPa |
| Binding liquid flow | 6 | g/min |
| volume | | |
| Airflow volume | 0.3 | m³/min |

| | |
|---|---|
| Granulation pattern | spraying for 30 sec, |
| | drying for 20 sec, and |
| | shaking for 10 sec |

### Granulation Conditions in Working Example 7

TABLE 10

| | | |
|---|---|---|
| Core | Crystalline cellulose (sphere) | 12.00 mg |
| First layer | Partially saponified polyvinyl alcohol | 0.36 mg |
| Second | Linaclotide | 0.0625 mg |
| layer | Calcium chloride | 0.175 mg |
| | L-Leucine | 0.08 mg |
| | Partially saponified polyvinyl alcohol | 0.16 mg |
| Third layer | Partially saponified polyvinyl alcohol | 0.385 mg |

TABLE 11

| | | |
|---|---|---|
| Granulation | Coated particles with third layer | 13.2225 mg |
| | D-Mannitol | 121.0275 mg |
| | Maltose | 7.50 mg |
| Mixing | Croscarmellose sodium | 7.50 mg |
| | Crystalline cellulose | 15.00 mg |
| | Magnesium stearate | 0.75 mg |
| Film coating | Opadry 85F42205 | 5.0 mg |

### (1) Preparing First Layer

1,000 g partially saponified polyvinyl alcohol was dissolved in 9,000 g purified water. 5,400 g of the partially saponified polyvinyl alcohol solution was sprayed onto 18,000 g of (granulated) crystalline cellulose (trade name: CP-102Y; Asahi Kasei Chemicals) using a Glatt GPCG-15 fluidized bed granulation apparatus (likewise for second layer) to prepare granules coated with a first layer.

### (2) Preparing Second Layer

A suitable amount of dilute hydrochloric acid (Kozakai Pharmaceutical) was added dropwise to 11,610 g purified water to adjust the pH to 1.5-2.0. After 227.5 g calcium chloride had been added thereto and completely dissolved, 81.25 g linaclotide was dissolved therein. After the linaclotide had completely dissolved, 104 g L-leucine was added thereto and completely dissolved. Separately, 208 g partially saponified polyvinyl alcohol was dissolved in 832 g purified water. The two solutions were mixed, after which dilute hydrochloric acid (Kozakai Pharmaceutical) was added dropwise thereto to adjust the pH to 1.5-2.0. The mixed fluid was sprayed onto 16,068 g of granules coated with the first layer using a fluidized bed granulation apparatus to prepare granules coated with a second layer.

### (3) Preparing Third Layer

500 g partially saponified polyvinyl alcohol was dissolved in 4,500 g purified water. 4,620 g of the partially saponified polyvinyl alcohol solution was sprayed onto 15,405 g of the granules coated with the second layer using a fluidized bed granulation apparatus to coat the granules with a third layer, thereby preparing granules coated with the third layer.

### (4) Granulation

750 g maltose was dissolved in 3,000 g purified water. Using a Glatt GPCG-15 fluidized bed granulation apparatus, 1,322.25 g of the granules coated with the third layer and 12,102.75 g D-mannitol were mixed in a fluidized bed, and the mixed product was sprayed with 3,750 g of the maltose solution at 260 g/min to create granules (product temperature: roughly 43° C.; spray pressure: 0.20 MPa; air flow rate: 7.5 m³/min). Granulation was performed using an intermittent spray method involving spraying for 15 seconds, drying for 35 seconds, and shaking for 15 seconds. The granulated product was dried until a product temperature of 50° C. was reached to obtain a granulated pharmaceutical composition containing the granular pharmaceutical composition according to the present invention.

### (5) Mixing, Tablet-Making

14,175 g of the granulated pharmaceutical composition containing the granular pharmaceutical composition, 750 g croscarmellose sodium (trade name: Kiccolate ND-2HS, Nichirin Chemical Industries; likewise hereafter), 1,500 g crystalline cellulose (trade name: Ceolus UF711, Asahi Kasei; likewise hereafter), and 75 g magnesium stearate were mixed using a polyethylene bag, after which the mixture was compression molded using a Hata Iron Works X-20 rotary tablet press to obtain tablets of a pharmaceutical composition containing the granular pharmaceutical composition according to the present invention.

### (6) Film Coating

1,000 g of a film coating agent (Opadry® 85F42205) was dispersed in 4,000 g purified water to prepare a film coating agent. Using a Freund Aqua Coater 48/60 60 film coating device, the tablets of the pharmaceutical composition containing the granular pharmaceutical composition according to the present invention were spray-coated with 500 g of the aqueous dispersion of the film coating agent to obtain tablets containing 170 mg apiece of the pharmaceutical composition containing the granular pharmaceutical composition according to the present invention.

### Working Example 9

TABLE 12

| | | | |
|---|---|---|---|
| Core | Crystalline cellulose (granular) | 12.00 | mg |
| First layer | Partially saponified polyvinyl alcohol | 0.36 | mg |
| Second layer | Linaclotide | 0.0625 | mg |
| | Partially saponified polyvinyl alcohol | 0.16 | mg |
| Third layer | Partially saponified polyvinyl alcohol | 0.377 | mg |

TABLE 13

| | | | |
|---|---|---|---|
| Granulation | Coated particles with third layer | 12.96 | mg |
| | D-Mannitol | 121.29 | mg |
| | Maltose | 7.5 | mg |
| Mixing | Croscarmellose sodium | 7.5 | mg |
| | Crystalline cellulose | 15.0 | mg |
| | Magnesium stearate | 0.75 | mg |

### (1) Preparing First Layer

300 g partially saponified polyvinyl alcohol was dissolved in 2,700 g purified water. 1,650 g of the partially saponified polyvinyl alcohol solution was sprayed onto 5,500 g of (granulated) crystalline cellulose (trade name: CP-102Y; Asahi Kasei Chemicals) using a Glatt GPCG-5 fluidized bed granulation apparatus (likewise for second layer) to prepare granules coated with a first layer.

### (2) Preparing Second Layer

25 g linaclotide was dispersed in 3,570 g purified water, after which a suitable amount of 10% hydrochloric acid (Wako Pure Chemicals) was added dropwise thereto to adjust the pH to 1.5-2.0. Separately, 64 g partially saponified polyvinyl alcohol was dissolved in 256 g purified water. After the ingredients had completely dissolved, the two liquids were mixed. The mixed fluid was sprayed onto 4,944 g of granules coated with the first layer using a fluidized bed granulation apparatus to prepare granules coated with a second layer.

### (3) Preparing Third Layer

200 g partially saponified polyvinyl alcohol was dissolved in 1,800 g purified water. 1,463 g of the partially saponified polyvinyl alcohol solution was sprayed onto 4,878 g of the granules coated with the second layer using a fluidized bed granulation apparatus to coat the granules with a third layer, thereby preparing granules coated with the third layer.

### (4) Granulation

240 g maltose was dissolved in 960 g purified water. Using a Glatt GPCG-5 fluidized bed granulation apparatus, 414.7 g of the granules coated with the third layer and 3,881.3 g D-mannitol were mixed in a fluidized bed, and the mixed product was sprayed with 1,200 g of the maltose solution at 100 g/minute to create granules (product temperature: roughly 40° C.; spray pressure: 0.2 MPa; air flow rate: 0.2 m³/min). Granulation was performed using an intermittent spray method involving spraying for 15 seconds, drying for 35 seconds, and shaking for 15 seconds. The granulated product was dried until a product temperature of 47° C. was reached to obtain a granulated pharmaceutical composition containing the granular pharmaceutical composition according to the present invention.

### (5) Mixing, Tablet-Making

4,252.5 g of the granulated pharmaceutical composition containing the granular pharmaceutical composition, 225 g croscarmellose sodium, 450 g crystalline cellulose, and 22.5 g magnesium stearate were mixed using a polyethylene bag, after which the mixture was compression molded using a Hata Iron Works X-20 rotary tablet press to obtain tablets of a pharmaceutical composition containing the granular pharmaceutical composition according to the present invention.

### Comparative Example 1

TABLE 14

| | | | |
|---|---|---|---|
| Core | Crystalline cellulose (particle) | 22.66 | mg |
| First layer | Linaclotide | 0.0625 | mg |
| | Calcium chloride | 0.361 | mg |
| | L-Leucine | 0.161 | mg |
| | Partially saponified polyvinyl alcohol | 0.164 | mg |

TABLE 15

| | | | |
|---|---|---|---|
| Granulation | Coated particles with first layer | 23.409 | mg |
| | D-Mannitol | 210.6 | mg |
| | Hypromellose | 7.0 | mg |
| Mixing | Crospovidone | 12.1 | mg |
| | Magnesium stearate | 1.3 | mg |

### (1) Preparing First Layer

100 g partially saponified polyvinyl alcohol was dissolved in 900 g purified water. 2.937 g linaclotide was dispersed in 363.9 g purified water, after which a suitable amount of 10% hydrochloric acid (Wako Pure Chemicals) was added dropwise thereto to adjust the pH to 1.5-2.0. Separately, 100 g partially saponified polyvinyl alcohol was dissolved in 900 g purified water. Separately, 7.56 g L-leucine and 16.95 g calcium chloride were dissolved in 198.9 g purified water, 77.0 g of the previously prepared partially saponified polyvinyl alcohol solution was added thereto, and 10% hydrochloric acid (Wako Pure Chemicals) was added dropwise thereto to adjust the pH to 1.5-2.0. After the ingredients had completely dissolved, the two liquids were mixed. 1.064.9 g of granulated crystalline cellulose (trade name: CP-102Ym, Asahi Kasei Chemicals) was sprayed with the mixed liquid using a fluidized bed granulation apparatus to prepare granules coated with a first layer.

### (2) Granulation

100 g hypromellose (trade name: TC-5E, Shin'etsu Chemical) was dissolved in 900 g purified water. 81.9 g of the granules coated with the third layer and 737.1 g D-mannitol were mixed in a fluidized bed, and the mixed product was sprayed with 245.0 g hypromellose at 6 g/min to create granules (product temperature: roughly 40° C.; spray pressure: 0.08 MPa; air flow rate: 0.2-0.3 m³/min). Granulation was performed using an intermittent spray method involving spraying for 30 seconds, drying for 20 seconds, and shaking for 10 seconds. The granulated product was dried until 48° C. was reached to obtain a comparative granulated pharmaceutical composition containing a granular pharmaceutical composition.

### (3) Mixing, Tablet-Making

482 g of the granulated pharmaceutical composition containing the granular pharmaceutical composition, 24.1 g crospovidone (trade name: XL-10, ISP), and 2.6 g magnesium stearate were mixed using a polyethylene bag, after which the mixture was compression molded using a Hata Iron Works X20 rotary tablet press to obtain tablets of a comparative pharmaceutical composition containing a granular pharmaceutical composition.

### Comparative Examples 2-5

TABLE 16

| | | | |
|---|---|---|---|
| Core | Crystalline cellulose (granular) | 22.66 | mg |
| First layer | Linaclotide | 0.0625 | mg |
| | Calcium chloride | 0.361 | mg |
| | L-Leucine | 0.161 | mg |
| | Hypromellose | 0.164 | mg |

TABLE 17

| | | | |
|---|---|---|---|
| Mixing | Coated particles with first layer | 23.4 | mg |
| | Various sugars or sugar alcohols | 211 | mg |

TABLE 18

| | |
|---|---|
| Comparative Example 2 | D-Mannitol |
| Comparative Example 3 | Lactitol |
| Comparative Example 4 | Maltitol |
| Comparative Example 5 | Trehalose |

### (1) Preparing First Layer

48.06 g linaclotide was dispersed in 7,601 g purified water, after which a suitable amount of 10% hydrochloric acid (Wako Pure Chemicals) was added dropwise thereto to adjust the pH to 1.5-2.0. Separately, 123.8 g L-leucine, 277.6 g calcium chloride, and 126.1 g hypromellose (HPMC) (trade name: TC-5E, Shin'etsu Chemical) were dissolved in 5,858 g purified water, and 10% hydrochloric acid (Wako Pure Chemicals) was added dropwise thereto to adjust the pH to 1.5-2.0. After the ingredients had completely dissolved, the two liquids were mixed. 17.425 kg granular crystalline cellulose (trade name: CP-102Y, Asahi Kasei Chemicals) was sprayed with the mixed liquid using a fluidized bed granulation apparatus form a coating of a first layer, thereby preparing a comparative granular pharmaceutical composition.

### (2) Mixing

23.4 mg of the granules coated with the first layer and 211 mg D-mannitol were introduced into a glass bottle, and the bottle was induction sealed to prepare a comparative pharmaceutical composition containing a granular pharmaceutical composition (comparative example 2).

Lactitol was substituted for the D-mannitol to prepare a pharmaceutical composition containing a granular pharmaceutical composition according to comparative example 3. Maltitol was substituted for the D-mannitol to prepare a pharmaceutical composition containing a granular pharmaceutical composition according to comparative example 4. Trehalose was substituted for the D-mannitol to prepare a pharmaceutical composition containing a granular pharmaceutical composition according to comparative example 5.

### Reference Example 1

Partially saponified polyvinyl alcohol (Gohsenol® EG-05P, Nippon Synthetic Chemical Industry) and 15 g ethyl cellulose (trade name: Aquacoat ECD, Dainippon Sumitomo) were dissolved in 250 mL ion-exchanged water to create a solution, which was poured into a Teflon® dish and dried at 40° C. for 24 hours. 15 g aminoacrylic methacrylate copolymer E (trade name: Eudragit E, EVONIK) was dissolved in 250 mL ethanol to create a solution, which was poured into a Teflon® dish and dried at 23° C. for 24 hours. A methacrylate copolymer LD stock solution (trade name: Eudragit L30 D55, EVONIK) was poured into the center of a aluminum plate having an outer diameter of 70 mm, an inner diameter of 10 mm, and a thickness of 1 mm and dried at 23° C. for 24 hours.

Using the obtained film, a moisture vapor transmission rate was performed at a temperature of 23° C.±1° C. and a humidity of 50±5% according to conditions A of JIS Z 0208:1976, “Method for testing the water vapor permeability of moisture-proof packaging materials (inverted cup method).”

Results for the moisture vapor transmission rate tests are shown in table 19.

TABLE 19

| | | | Water vapor |
|---|---|---|---|
| | Permeation | Average | transmission |
| Samples | rate | thickness | rate (g/(m² · h)) |
| Partially saponified | 28.26 mm² | 0.30 mm | 1.2 |
| polyvinyl alcohol | | | |
| Ethyl cellulose | 28.26 mm² | 0.25 mm | 1.1 |
| Aminoalkyl methacrylate | 12.56 mm² | 0.28 mm | 1.0 |
| copolymer E | | | |
| Methacrylic acid | 0.785 mm² | 0.47 mm | 17 |
| copolymer LD | | | |

### Test Example 1

10 g of the granular pharmaceutical composition according to working example 1 was sealed into a two-ply polybag, which was sealed in an aluminum pouch and left standing at a temperature of 40° C. and 75% RH for 6 months.

The amount of degradation product after storage was measured using high-speed liquid chromatography. A YMC-PackPro® C18 column (dimensions: 3.0×150 mm, 3.0 urn; YMC) or a comparable product was used, and the temperature was maintained at 40° C. An eluant A (MPA) was constituted by 2% acetonitrile to 98% water and 0.1% trifluoroacetic acid, and an eluant B (MPB) was constituted by 95% acetonitrile to 5% water and 0.1% trifluoroacetic acid. Degradation products were eluted at a gradient of 0% in 4 minutes, from 0% to 10% MPB in 9 minutes, from 10% to 23% MPB in 43 minutes, from 23% to 34% MPB in 49 minutes, from 34% to 80% MPB in 59 minutes, from 80% to 0% MPB in 60 minutes, and 0% MPB in 67 minutes. The flow rate was 0.6 mL/minute, and detection was performed using 220 nm UV light. An analysis specimen was prepared by adding a predetermined amount of the granulated product of tablets to 0.1 N hydrochloric acid to a target concentration of 0.2 μg linaclotide/mL. 100 mL of the solution was injected into the column.

The linaclotide concentration of the prepared specimen was measured against a similarly prepared linaclotide external reference to determine the linaclotide content.

“Cys¹-IMD” in table 20 indicates linaclotide formaldehyde imidazolidinone products at a retention time of 1.12 minutes. “Cys¹-Ketone” in table 20 indicates linaclotide degradation products at a retention time of 1.18 minutes.

The total amount of degradation products and the proportion of the primary degradation products (retention times: 1.12 minutes and 1.18 minutes) against the total amount of linaclotide and degradation products thereof is shown in table 20.

TABLE 20

| | Total degradation | | |
|---|---|---|---|
| | products | Cys¹-IMD | Cys¹-Ketone |
| | (surface area %) | (surface area %) | (surface area %) |
| Working | 1.93 | 0.07 | 0.34 |
| Example 1 | | | |

### Test Example 2

One capsule from working example 1 was sealed in an aluminum-aluminum blister (double-chambered; one desiccant package containing granular silica gel), and left standing at a temperature of 40° C. and a humidity of 75% RH for three months.

The amount of degradation product after storage was measured using high-speed liquid chromatography. The total amount of degradation products and the proportion of the primary degradation products (retention times: 1.12 minutes and 1.18 minutes) against the total amount of obtained linaclotide and degradation products thereof is shown in table 21.

TABLE 21

| | Total degradation | | |
|---|---|---|---|
| | products | Cys¹-IMD | Cys¹-Ketone |
| | (surface area %) | (surface area %) | (surface area %) |
| Working | 1.05 | N.D. | 0.10 |
| Example 1 | | | |

N.D.: Not Determined

### Test Example 3

One tablet each from working example 2 and comparative example 1 were packed in four-way MoistCatch® packages, which were left standing at 60° C. and ambient humidity for 21 days.

The total amount of degradation products and the proportion of the primary degradation products (retention times: 1.12 minutes and 1.18 minutes) against the total amount of linaclotide and degradation products thereof is shown in table 22.

TABLE 22

| | | Total degradation | | |
|---|---|---|---|---|
| | | products | Cys¹-IMD | Cys¹-Ketone |
| | | (surface | (surface | (surface |
| | | area %) | area %) | area %) |
| 60° C., | Working | 1.22 | 0.07 | 0.11 |
| ambient | Example 2 | | | |
| humidity, | Comparative | 9.24 | 2.06 | 0.84 |
| 21 days | Example 1 | | | |

### Test Example 4

Mixtures of granules from working examples 3-6 and comparative examples 2-5 with a sugar or sugar alcohol were sealed in glass bottles and stored at a temperature of 40° C. and 75% RH for three months.

The amount of degradation product after storage was measured using high-speed liquid chromatography. The total amount of degradation products and the proportion of the primary degradation products (retention times: 1.12 minutes and 1.18 minutes) against the total amount of obtained linaclotide and degradation products thereof is shown in table 23.

TABLE 23

| | | Total degradation | | |
|---|---|---|---|---|
| | | products | Cys¹-IMD | Cys¹-Ketone |
| | | (surface | (surface | (surface |
| | | area %) | area %) | area %) |
| D-Mannitol | Working | 2.85 | 0.26 | 0.54 |
| | Example 3 | | | |
| | Comparative | 28.50 | 2.90 | 19.57 |
| | Example 2 | | | |
| Lactitol | Working | 2.44 | 0.22 | 0.18 |
| | Example 4 | | | |
| | Comparative | 16.66 | 2.63 | 8.00 |
| | Example 3 | | | |
| Maltitol | Working | 2.72 | 0.12 | 0.21 |
| | Example 5 | | | |
| | Comparative | 11.21 | 2.15 | 4.04 |
| | Example 4 | | | |
| Trehalose | Working | 2.30 | 0.25 | 0.28 |
| | Example 6 | | | |
| | Comparative | 27.88 | 2.20 | 19.87 |
| | Example 5 | | | |

### Test Example 5

The granulated product from working example 7 was sealed in a four-way MoistCatch® package and left standing at a temperature of 40° C. and a humidity of 75% RH for two months.

The amount of degradation product after storage was measured using high-speed liquid chromatography. The total amount of degradation products and the proportion of the primary degradation products (retention times: 1.12 minutes and 1.18 minutes) against the total amount of obtained linaclotide and degradation products thereof is shown in table 24.

TABLE 24

| | Total degradation | | |
|---|---|---|---|
| | products | Cys¹-IMD | Cys¹-Ketone |
| | (surface area %) | (surface area %) | (surface area %) |
| Working | 3.22 | 1.06 | 0.28 |
| Example 7 | | | |

### Test Example 6

One tablet from working example 8 was sealed in a four-way MoistCatch® package and left standing at a temperature of 40° C. and a humidity of 75% RH for six months.

The amount of degradation product after storage was measured using high-speed liquid chromatography. The total amount of degradation products and the proportion of the primary degradation products (retention times: 1.12 minutes and 1.18 minutes) against the total amount of obtained linaclotide and degradation products thereof is shown in table 25.

TABLE 25

| | Total degradation | | |
|---|---|---|---|
| | products | Cys¹-IMD | Cys¹-Ketone |
| | (surface area %) | (surface area %) | (surface area %) |
| Working | 1.24 | 0.23 | 0.18 |
| Example 8 | | | |

### Test Example 7

One tablet from working example 9 was sealed in a four-way MoistCatch® package and left standing at a temperature of 40° C. and a humidity of 75% RH for three months.

The amount of degradation product after storage was measured using high-speed liquid chromatography. The total amount of degradation products and the proportion of the primary degradation products (retention times: 1.12 minutes and 1.18 minutes) against the total amount of obtained linaclotide and degradation products thereof is shown in table 26.

TABLE 26

| | Total degradation | | |
|---|---|---|---|
| | products | Cys¹-IMD | Cys¹-Ketone |
| | (surface area %) | (surface area %) | (surface area %) |
| Working | 1.50 | <0.05% | <0.05% |
| Example 9 | | | |

### INDUSTRIAL APPLICABILITY

In accordance with the present invention, a linaclotide-containing granular pharmaceutical composition of improved stability, especially a tablet containing said granular pharmaceutical composition, can be provided.

The foregoing has been a description of specific aspects of the present invention, but various modifications and alterations that will be obvious to a person skilled in the art are comprehended within the scope of the present invention.

### Sequence Table Free Text

The base sequence of sequence No. 1 in the sequence table is a synthetic peptide.

1. A granular pharmaceutical composition obtained by coating a core with (1) a layer containing a substance exhibiting moisture-proofing action, (2) a drug layer containing linaclotide, a pharmaceutically acceptable salt thereof, or a hydrate of either, and (3) a layer containing a substance exhibiting moisture-proofing action.

2. The granular pharmaceutical composition according to claim 1, wherein the layer (1) or (3) containing a substance exhibiting moisture-proofing action contains a substance having a moisture vapor transmission rate of no more than 20 g/(m²·h) as the substance exhibiting moisture-proofing action.

3. The granular pharmaceutical composition according to claim 1 or 2, wherein the layer (1) or (3) containing a substance exhibiting moisture-proofing action contains one or more substances selected from the group consisting of polyvinyl alcohol, methacrylate copolymer S, PVA copolymer, aminoalkyl methacrylate copolymer E, methacrylate copolymer LD, and ethyl-cellulose as the substance exhibiting moisture-proofing action.

4. The granular pharmaceutical composition according to any one of claims 1-3, wherein the layer (1) or (3) containing a substance exhibiting moisture-proofing action contains one or more substances selected from the group consisting of polyvinyl alcohol, methacrylate copolymer S, and PVA copolymer as the substance exhibiting moisture-proofing action.

5. The granular pharmaceutical composition according to any one of claims 1-4, wherein the composition contains at least 100% by weight and no more than 50,000% by weight of the substance exhibiting moisture-proofing action with respect to the weight of the linaclotide, pharmaceutically acceptable salt thereof, or hydrate of either.

6. The granular pharmaceutical composition according to any one of claims 1-5, wherein the composition contains at least 0.5% by weight and no more than 30% by weight of the substance exhibiting moisture-proofing action with respect to the weight of the core.

7. A drug preparation containing the granular pharmaceutical composition according to any one of claims 1-6, the preparation being obtained via granulation.

8. The drug preparation according to claim 7, wherein the drug preparation contains at least 2.7 μg and no more than 6 mg of linaclotide.

9. The drug preparation according to claim 7 or 8, wherein the drug preparation contains one or more substances selected from the group consisting of hydroxypropyl cellulose, hydroxypropyl methyl cellulose, polyvinyl alcohol, PVA copolymer, trehalose, sorbitol, lactitol, isomalt, maltose, oligosaccharides, and maltitol as a binder used for granulation.

10. The drug preparation according to any one of claims 7-9, wherein the granulated product has a moisture content of at least 0.3% and no more than 4%.

11. The drug preparation according to any one of claims 7-10, wherein the drug preparation is selected from the group consisting of powders, fine granules, dry syrups, capsules, tablets, orally disintegrating tablets, pills, and troches.

12. The granular pharmaceutical composition or drug preparation according to any one of claims 1-11, wherein the total amount of degradation products with respect to the linaclotide, pharmaceutically acceptable salt thereof, or hydrate of either, or degradation products thereof, is no more than 8%, the total amount of Cys¹-IMD is no more than 2%, and the total amount of Cys¹-Ketone is no more than 2%.

13. A method of producing a granular pharmaceutical composition, the method comprising (1) a step of coating a core with a layer containing a substance exhibiting moisture-proofing action, (2) a step of coating with a drug layer containing linaclotide, a pharmaceutically acceptable salt thereof, or a hydrate of either, and (3) a step of coating with a layer containing a substance exhibiting moisture-proofing action.

14. The method of producing a granular pharmaceutical composition according to claim 13, wherein the layer (1) or (3) containing a substance exhibiting moisture-proofing action contains a substance having a moisture vapor transmission rate of no more than 20 g/(m²·h) as the substance exhibiting moisture-proofing action.

15. The method of producing a granular pharmaceutical composition according to claim 13 or 14, wherein the layer (1) or (3) containing a substance exhibiting moisture-proofing action contains one or more substances selected from the group consisting of polyvinyl alcohol, methacrylate copolymer S, PVA copolymer, aminoalkyl methacrylate copolymer E, methacrylate copolymer LD, and ethyl-cellulose as the substance exhibiting moisture-proofing action.

16. The method of producing a granular pharmaceutical composition according to any one of claims 13-15, wherein the layer (1) or (3) containing a substance exhibiting moisture-proofing action contains one or more substances selected from the group consisting of polyvinyl alcohol, methacrylate copolymer S, and PVA copolymer as the substance exhibiting moisture-proofing action.

17. The method of producing a granular pharmaceutical composition according to any one of claims 13-16, wherein the composition contains at least 100% by weight and no more than 50,000% by weight of the substance exhibiting moisture-proofing action with respect to the weight of the linaclotide, pharmaceutically acceptable salt thereof, or hydrate of either.

18. The method of producing a granular pharmaceutical composition according to any one of claims 13-17, wherein the composition contains at least 0.5% by weight and no more than 30% by weight of the substance exhibiting moisture-proofing action with respect to the weight of the core.

19. A method of producing a drug preparation containing the granular pharmaceutical composition according to any one of claims 13-18, the method further containing a granulation step.

20. The method of producing a drug preparation according to claim 19, wherein the drug preparation contains at least 2.7 μg and no more than 6 mg of linaclotide.

21. The method of producing a drug preparation according to claim 19 or 20, wherein the drug preparation contains one or more substances selected from the group consisting of hydroxypropyl cellulose, hydroxypropyl methyl cellulose, polyvinyl alcohol, PVA copolymer, trehalose, sorbitol, lactitol, isomalt, maltose, oligosaccharides, and maltitol as a binder used for granulation.

22. The method of producing a drug preparation according to any one of claims 19-21, wherein granules are formed via an intermittent spray method.

23. The method of producing a drug preparation according to any one of claims 19-22, wherein the granulated product has a moisture content of at least 0.3% and no more than 4%.
